(19) United States
(12) Patent Application Publication
(10) Pub. No.: US 2017/0079848 A1
(43) Pub. Date: Mar. 23, 2017

(54) METHOD FOR MAKING ABSORBENT ARTICLES

(71) Applicant: GDM S.p.A., Bologna (IT)

(72) Inventors: Matteo PIANTONI, Albino (Bergamo) (IT); Valerio SOLI, Bologna (IT)

(21) Appl. No.: 15/312,263

(22) PCT Filed: May 21, 2014
(86) PCT No.: PCT/IB2014/061596

§ 371 Date: Nov. 18, 2016

(51) Int. Cl. A61F 13/15; B29C 65/48

(52) U.S. Cl. CPC A61F 13/15723; A61F 13/15764; A61F 13/15699; B29C 65/48; B29L 2031/4878

(57) ABSTRACT

A method for making absorbent sanitary articles which include a permeable inner layer, an impermeable outer layer and an absorbent pad enclosed between them; the absorbent pad includes a first bottom layer and a second top layer between which an absorbent core is enclosed; the method includes a step of forming the absorbent pad; the step of forming the absorbent pad includes a step of feeding a first continuous web, which will define the first bottom layer and a second continuous web, which will define the second top layer, and a step of interposing between the first web and the second web at least a third web which has a plurality of openings and which will define the absorbent core; the method includes a step of feeding a third continuous web along a feed direction and a step of cutting the third continuous web to obtain the openings.

### TECHNICAL FIELD

This invention relates to a method for making absorbent articles.

More specifically, this invention relates to a method for making an absorbent pad of the absorbent article.

### BACKGROUND ART

Typically, nappies comprise an impermeable layer, for example of polyethylene, a layer of non-woven fabric permeable to liquids and an absorbent pad sandwiched therebetween.

In recent years, in order to reduce the cost of nappies while maintaining a good absorption capacity of the padding, absorbent pads have been developed which are known as “fluffless” since they do not contain absorbent material made from cellulose fibre (“fluff”), but contain only superabsorbent polymer material.

These absorbent pads are composed of a first and a second layer of non-woven fabric joined to each other in such a way as to form a plurality of cells containing the superabsorbent polymer material.

More specifically, the cells are formed by sealing together the first and second layers of non-woven fabric according to a predetermined pattern, usually grid-like.

Also known from the prior art are fluffless absorbent pads having a third layer of permeable material interposed between the first and the second layer of non-woven fabric having a plurality of openings or cells in which the superabsorbent polymer material is placed.

The presence of a third layer provided with the aforementioned cells makes it possible to do away with the seals between the first and second layers of non-woven fabric, thereby rendering the method for forming the absorbent pad simpler and more economical.

The third layer of permeable material is usually purchased by the nappy manufacturers as a finished product which has the aforementioned openings ready made in it and whose cost is therefore relatively high, considerably raising the end cost of the nappy.

### AIM OF THE INVENTION

This invention has for an aim to provide a method for making absorbent articles which is easy to implement compared to the prior art so as to reduce the costs connected with the absorbent pad, as set out in independent claim 1.

### BRIEF DESCRIPTION OF THE DRAWINGS

The invention will now be described with reference to the accompanying drawings which illustrate non-limiting embodiments of it and in which:

FIG. 1 shows a schematic front view of a first embodiment of a device for making absorbent articles and capable of implementing the method according to the invention;

FIG. 2 shows a schematic front view of a second embodiment of a device for making absorbent articles and capable of implementing the method according to the invention;

FIG. 3 shows a schematic plan view of a first embodiment of an absorbent article made according to the method of the invention;

FIG. 4 shows a schematic plan view of a second embodiment of an absorbent article made according to the method of the invention.

### DETAILED DESCRIPTION OF PREFERRED EMBODIMENTS OF THE INVENTION

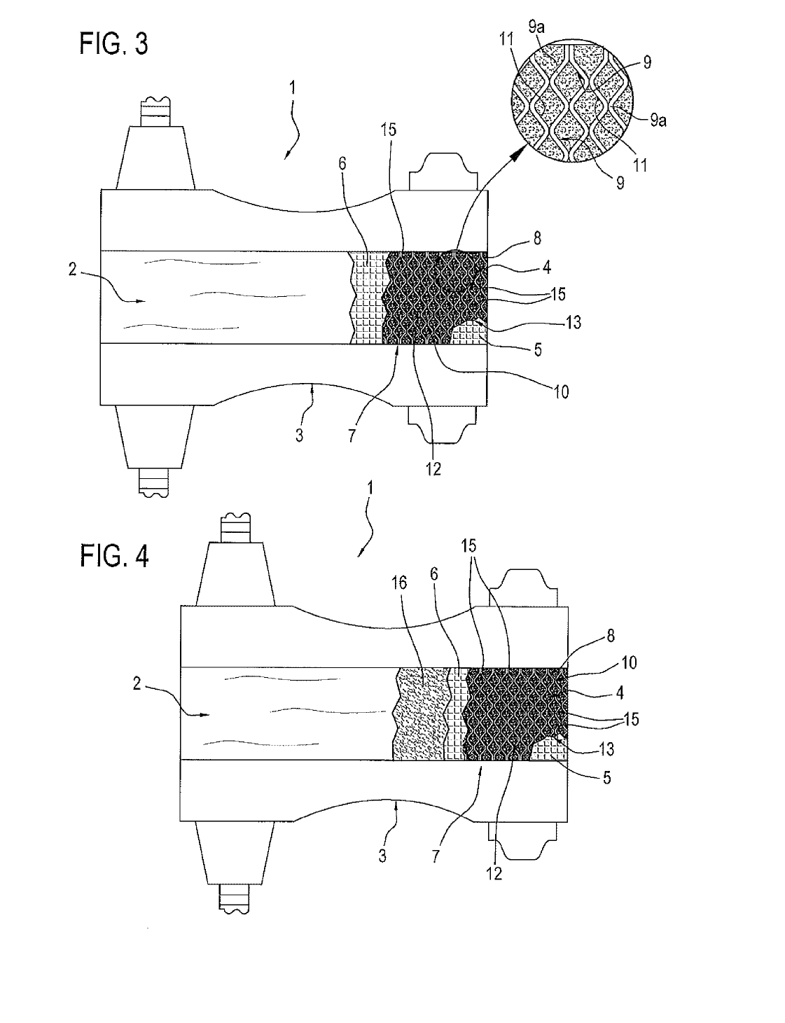

With reference to the accompanying drawings, in particular FIGS. 3 and 4, the numeral 1 denotes an absorbent article according to the invention, such as, for example, a nappy to which this specification refers but without thereby limiting the scope of the invention.

The article 1 comprises an inner layer 2 permeable to liquids, also known as “top sheet”, preferably defined by a piece of non-woven fabric, and an outer layer 3 impermeable to liquids, also known as “back sheet”, preferably defined by a sheet of polyethylene.

The inner layer 2 and the outer layer 3 are defined as such with reference to the side of the absorbent article in contact with the wearer's skin.

The article 1 comprises an absorbent pad 4 interposed between the permeable inner layer 2 and the impermeable outer layer 3.

The absorbent pad 4 comprises a first bottom layer 5 and a second top layer 6 between which an absorbent core 7 is enclosed.

The first bottom layer 5 and the second top layer 6 are joined to each other as described in more detail below.

Preferably, the first bottom layer 5 and the second top layer 6 are sheets of non-woven fabric.

More specifically, the first bottom layer 5 is positioned to face the impermeable outer layer 3 and is preferably in contact therewith.

Preferably, the first bottom layer 5 is connected to the impermeable outer layer 3 by an adhesive-like substance.

The second top layer 6 faces the permeable inner layer 2 and is preferably in contact therewith.

Preferably, the second top layer 6 is connected to the permeable inner layer 2 by an adhesive-like substance.

The pad 4 comprises an absorbent core 7 interposed between the first layer 5 and the second layer 6.

The absorbent core 7 comprises at least a third layer 8 preferably of permeable material.

The third layer 8 is preferably made from a non-woven fabric with a spongy consistency, known as “foam”.

In a further preferred embodiment, the third layer 8 is preferably made from a non-woven fabric known as “high loft”, which is an extruded non-woven fabric of substantially known type and composed typically of polypropylene and polyethylene.

The third layer 8 has a plurality of openings 9.

Preferably, the openings 9 are through openings and have respective delimiting edges 9a defining a mesh 10.

Advantageously, the third layer 8 is made from a permeable material allowing fluid to pass from one opening 9 to another and to be distributed along the whole of the pad 4, even to the most peripheral zones.

The edges 9a of the openings 9 have side walls 11, a front face 12 and a rear face 13.

The front face 12 and the rear face 13 of all the edges 9a of the openings 9 define the front face 12 and the rear face 13 of the third layer 8.

The front face 12 of the edges 9a is in contact with the second top layer 6 of the pad 4.

The rear face 13 is in contact with the first bottom layer 5.

Preferably, at least one between the front face 12 and the rear face 13 is connected to the second top layer 6 and to the first bottom layer 5, respectively.

Advantageously, either the front face 12 or the rear face 13 is connected to the second top layer 6 or to the first bottom layer 5, respectively.

Preferably, a layer of adhesive substance 14 is interposed between the front face 12 and the second top layer 6.

Preferably, a layer of adhesive substance 14 is interposed between the rear face 13 and the first bottom layer 5.

In an alternative preferred embodiment, the first and second bottom and top layers 5 and 6 are connected to each other and the third layer 8 is enclosed between them without being connected either to the first layer 5 or to the second layer 6.

In other words, in this embodiment, the front face 12 and the rear face 13 of the third layer 8 are in contact with the second top layer 6 and with the first bottom layer 5 of the pad 4, respectively, without having the layer of adhesive substance 14 interposed between them.

It should be noted that the third layer 8 is a three-dimensional third layer having a non-negligible thickness in a direction transversal to the main longitudinal direction of extension of the third layer 8 itself.

The thickness of the third layer 8 substantially defines the thickness of the absorbent pad 4 as a whole.

The connection between the first and second bottom and top layers 5 and 6 may be made by sealing or by means of an adhesive-like substance.

The absorbent pad 4 comprises inside it a spread 15 of absorbent material, preferably a polymer known as superabsorbent polymer or SAP, to which express reference is made herein without thereby limiting the invention.

More specifically, the absorbent material 15 is located inside the openings 9 of the mesh 10 of the third layer 8, preferably in each of the openings 9 of the mesh 10, thus defining with the third layer 8 the absorbent core 7.

Advantageously, the absorbent material 15 maintains its position inside the respective opening 9 of the third layer 8 thanks to the containing action of the side walls 11 of the respective edges 9a and of the first layer 5 and second layer 6 in contact with the rear face 13 and front face 12 of the third layer 8.

The spread of absorbent polymer material 15 comprises at least a first absorbent polymer material.

In an alternative embodiment, the spread of absorbent polymer material 15 comprises a first absorbent polymer material and a second absorbent polymer material.

Advantageously, the liquid absorption reaction time of the second material is, generally speaking, longer than the liquid absorption reaction time of the first material.

In other words, the first material starts absorbing liquid before the second material which starts absorbing the liquid at a later stage.

Thus, during the initial stages of absorption, the second absorbent material leaves the flowability of the liquid substantially unaffected, allowing the liquid to reach the first absorbent material, which starts absorbing it immediately.

In the alternative embodiment illustrated in FIG. 4, the absorbent core 7 comprises at least a fourth layer 16 interposed between the third layer 8 and the second top layer 6 or interposed between the third layer 8 and the first bottom layer 5.

It should be noted that if the fourth layer 16 is interposed between the third layer 8 and the second top layer 6, the third layer 10 is in contact with the first bottom layer 5.

Alternatively, if the fourth layer 16 is interposed between the third layer 8 and the first bottom layer 5, the third layer 10 is in contact with the second top layer 6.

Preferably, the fourth layer 16 of non-woven fabric is a piece of a continuous web of non-woven fabric.

The fourth layer 16 is preferably made from a non-woven fabric known as “high loft”, which is an extruded non-woven fabric of substantially known type and composed typically of polypropylene and polyethylene, to which express reference is made herein without thereby limiting the invention.

In one embodiment, the fourth layer 16 extends substantially for the full length of the pad 4.

In a different embodiment, the fourth layer 16 of non-woven fabric is not continuous, that is, it is in the form of two or more pieces.

In one embodiment, the fourth layer 16 is as long as the entire pad 4 or, alternatively, is shorter than the pad 4 and preferably located in a median portion of the pad.

In addition to the above mentioned basic components, the absorbent articles 1 comprise further accessory components, such as, for example, elastic bands, faecal barriers and side tabs, which vary the complexity of the structure and production process of the absorbent articles to different degrees and which are not described here because they do not form part of this invention.

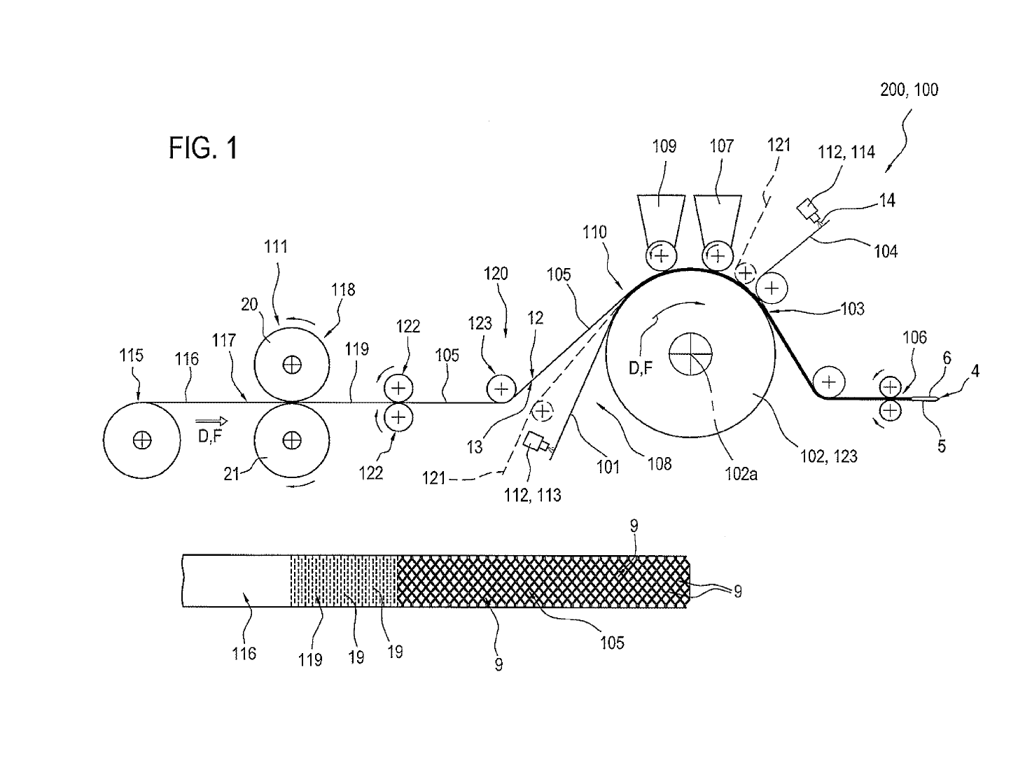

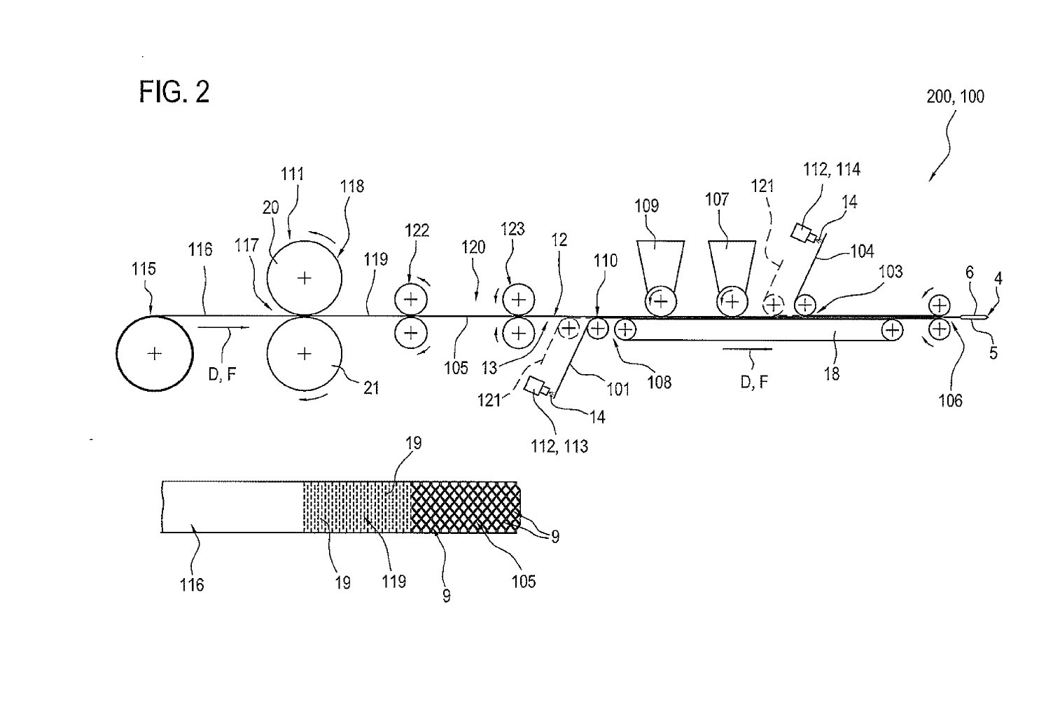

With reference to FIGS. 1 to 2, the numeral 200 denotes a machine for making the absorbent sanitary articles 1.

The description of the machine 200 is limited to the parts necessary for understanding this invention and, in particular, to the technical features which distinguish the different embodiments of it from each other, it being understood that the other features are the same for all the embodiments and denoted by the same reference numerals.

The machine 200 comprises a line, not illustrated, for feeding at least one continuous web of impermeable material used to make the impermeable outer layer 3.

The machine 200 comprises a forming unit 100 for making the absorbent pads 4 to form a succession of the pads 4 and to associate them with the continuous web of impermeable material.

The unit 100 comprises a system 108 for feeding a first web 101 of non-woven fabric to define the first bottom layer 5 of non-woven fabric.

The first web 101 advances in a predetermined feed direction D indicated by the arrow F in FIGS. 1 and 2.

The unit 100 comprises a first station 110 for positioning on the first web 101 of non-woven fabric a third web 105 which has a plurality of openings 9 and which will define the absorbent core 7.

The unit 100 comprises a second station 103 for positioning a second web 104 of non-woven fabric to define the second top layer 6 of non-woven fabric of the pad 4. At the second positioning station 103, the second web 104 of non-woven fabric is positioned on the third web 105.

The second station 103 for positioning the second web 104 of non-woven fabric is located downstream of the first positioning station 105 along the feed direction D of the first web 101 of non-woven fabric.

The unit 100 comprises a first station 109 for depositing the first spread of absorbent polymer material 15 on the third web 105 of non-woven fabric, and more specifically, for depositing the absorbent polymer material 15 inside the openings 9 of the mesh 10.

The first depositing station 109 is operatively located upstream of the second station 103 for positioning the second web 104 on the third web 105 and downstream of the first station 110 for positioning the third web 105 on the first web 101

The second positioning station 103 is located downstream of the first depositing station 109 in the sense that it operates on the third web 105 to apply thereon the second web 104 which, as already mentioned, will define the second top layer 6 of the pad 4 after the first station 109 has deposited the absorbent material 15.

If the spread of absorbent polymer material 15 of the pad 4 comprises a first absorbent polymer material and a second absorbent polymer material, the unit 100 comprises the first station 109 for depositing the first absorbent material and, downstream of the first depositing station 109, a second station 107 for depositing the second absorbent polymer material on the third web 105 and, more specifically, for depositing the second absorbent polymer material in the openings 9 of the mesh 10.

The unit 100 comprises a station 106 for joining the second web 104 to the first web 101 so as to close the third web 105 and the absorbent material 15 between them, thereby defining the absorbent core 7.

In order to connect the second web 104 to the first web 101, the unit 100 comprises at least one applicator 112 for applying an adhesive substance of the first web 101 and/or of the second web 104.

Preferably, at the joining station 106, the second web 104 is connected both to the first web 101 and to the third web 105 having the openings 9.

The applicator is located upstream of the joining station 106.

Preferably, the unit 100 comprises a first applicator 113 for applying an adhesive substance of the first web 101 and a second applicator 114 for applying an adhesive substance of the second web 104.

The first and second applicators 113 and 114 are located, respectively, upstream and downstream of the second positioning station 110 and 103.

Thus, at the first positioning station 110, the first web 101 is connected to the third web 105 having the openings 9, and more specifically, to the rear face 13 of the third web 105.

At the second positioning station 103, the second web 104 is connected to the third web 105 having the openings 9, and more specifically, to the front face 12 of the third web 105.

It should be noted that the joining station 106 might coincide with the second positioning station 103 both in the event that only the first web 101 and the second web 104 are connected to each other and in the event that the first and third webs 101 and 105 are connected to the second web 104.

Alternatively, the joining station 106 is located downstream of the second positioning station 103.

The first and second embodiments of the unit 100 differ in that in the first embodiment the unit 100 comprises a drum 102 for forming and feeding the succession of absorbent pads 4, whereas in the second embodiment, the unit 100 comprises a line 18 for forming and feeding the succession of absorbent pads 4.

According to the invention, the unit 100 comprises a station 115 for unwinding a third continuous web 116 which will define the third web 105 having the openings 9.

From the unwinding station 115, the third continuous web 116 advances in a direction parallel to the predetermined feed direction D, as indicated by the arrow F.

A cutting station 111 is located downstream of the station 115 for unwinding the third continuous web 116.

In a preferred embodiment, at the cutting station 111, cutting means 118 are provided for making a plurality of slits 19 on the third continuous web 116.

The cutting means 118 comprise mechanical or laser cutting elements.

In the embodiment illustrated, the cutting means 118 comprise a first cutting roller 20 and a second opposing element 21.

The second opposing element 21 is preferably in the form of an opposing roller 21, as illustrated in FIGS. 1 and 2, or in the form of a conveyor belt, not illustrated.

The first roller 20 has on its mantle a plurality of blades for cutting slits in the third continuous web 116 .

The first cutting roller 20 makes the slits 19 suitably oriented on the third continuous web 116.

Preferably, the slits 19 are made by cutting means 118 on the third continuous web 116 in a direction transversal to the feed direction D of the third continuous web 116.

Alternatively, the slits 19 are made by cutting means 118 on the third continuous web 116 in a direction parallel to the feed direction D of the third continuous web 116.

In a further variant embodiment, the slits 19 are made by the cutting means 118 on the third continuous web 116 according to a predetermined geometrical shape.

In an alternative embodiment not illustrated, the cutting means 118 cut defined portions out of the third continuous web 116 and remove these portions to form the third web 105 having the openings 9.

It should be noted that the position and shape of the cutting blades of the first cutting roller 20 is a function of the type of slit to be made on the third continuous web 116.

After slits have been cut in it, the third cut web 119 is fed out from the cutting station 111.

The unit 100 comprises a stretching station 120 for the third cut web 119, at which the third cut web 119 is stretched in such a way as to make the third web 105 having the openings 9.

The third web 105 having the openings 9 is fed out from the stretching station 120 towards the first positioning station 110.

At the stretching station 120, the unit 100 comprises at least two pairs of stretching rollers 122 and 123, and more specifically, a first and a second pair of stretching rollers 122 and 123.

The third cut web 119 is fed between the two rollers of each pair 122 and 123.

In order to stretch the third cut web 119, the second pair 123 of stretching rollers rotate at a higher speed than the first pair 122 in such a way that the third cut web 119 is elongated.

The third web 105 having the openings 9 is fed out from the second pair 123 of rollers towards the first positioning station 110.

It should be noted that in the first embodiment of the unit 100, illustrated in FIG. 1, one of the rollers of the second pair 123 is the forming and feeding drum 102.

In the preferred embodiment, the cut web 119 is stretched in a direction parallel to the feed direction D.

Alternatively, the cut web 119 is stretched in a direction transversal to the feed direction D.

In a further variant embodiment, the cut web 119 is stretched in a direction parallel and transversal to the feed direction D.

In order to make the openings 9 of the mesh 10, the slits 19 on the third continuous web 116 are best made in a direction parallel or transversal to the feed direction D as a function of the direction in which the cut web 119 is stretched.

Advantageously, if the slits in the continuous web 116 are cut along a direction transversal to the feed direction D, the cut web 119 is stretched in a direction parallel to the feed direction D.

If the slits in the continuous web 116 are cut along a direction parallel to the feed direction D, the cut web 119 is stretched in a direction transversal to the feed direction D.

If the slits in the continuous web 116 are cut according to a defined geometry, for example in the shape of a cross, the cut web 119 is stretched in a direction parallel and transversal to the feed direction D.

It should be noted that the stretching force applied to the cut web 119 determines the thickness of the third web 105 having the openings 9 and, consequently, of the third layer 8.

Varying the stretching force varies the thickness of the third web 105 having the openings 9.

Preferably, the thickness of the third web 105 having the openings 9 and, consequently, of the third layer 8, is constant along a direction parallel and transversal to the feed direction D.

Alternatively, varying the stretching force allows varying the thickness of the third web 105 along a direction parallel and/or transversal to the feed direction D.

Also forming an object of this invention is the method for making absorbent articles which comprises a step of forming the absorbent pad 4 as described in the foregoing and which is also described only insofar as necessary to understand the invention.

The method comprises a step of feeding a first continuous web 101 which will define the first bottom layer 5, and a second continuous web 104 which will define the second top layer 6, and a step of interposing between the first web 101 and the second continuous web 104 at least a third web 105 having the openings 9.

To make the third web 105 having the openings 9, the method comprises a step of feeding a third continuous web 116 and a step of cutting a plurality of slits 19 in the third continuous web 105 to obtain a third cut web 119.

In a first variant embodiment, the plurality of slits 19 in the third continuous web 105 are cut at least in a direction parallel to the feed direction D of the third continuous web 105.

In a preferred variant embodiment, the plurality of slits 19 in the third continuous web 105 are cut at least in a direction transversal to the feed direction D of the third continuous web 105.

The method comprises a step of stretching the third cut web 119 to make the third web 105 having the openings 9.

The step of stretching the third continuous cut web 119 is performed at least along a direction parallel to the feed direction D of the third continuous web 119.

Alternatively, the step of stretching the third continuous cut web 119 is performed at least along a direction transversal to the feed direction D of the third continuous web 119.

In a further variant embodiment, the step of stretching the third continuous cut web 119 is performed at least along a direction parallel and transversal to the feed direction D of the third continuous web 119.

In an alternative embodiment not illustrated, the method comprises a step of cutting defined portions out of the third continuous web 116 and removing these portions to form the third web 105 having the openings 9.

To connect at least the first continuous web 101 to the second continuous web 104, the method comprises a step of joining at least the first and the second continuous web 101 and 104 to the third web 105 having the openings 9 interposed between the first and the second continuous web 101 and 104.

In the preferred embodiment, the third web 105 having the openings 9 is connected to at least one of either the first continuous web 101 or to the second continuous web 104.

Consequently, the method comprises a step of applying a layer of adhesive substance 14 at least on the first continuous web 101 before the step of interposing the third web 105 having the openings 9 and the step of joining the third web 105 at least to the first continuous web 101.

In order to connect the third web 105 having the openings 9 also or only to the second continuous web 104, the method comprises a step of applying a layer of adhesive substance 14 at least on the second continuous web 104 before the step of interposing the third web 105.

In order to make the absorbent core 7, the method comprises a step of depositing at least one highly absorbent material 15 in the openings 9 of the mesh 10 of the third web 105.

Next, the second continuous web 104 is positioned on the third web 105 having the openings 9 with the highly absorbent material 15 in them.

If the absorbent pad 4 comprises the fourth layer 16 interposed between the third layer 8 and the second top layer 6, the method comprises a step of positioning a fourth continuous web 121 between the third web 105, having the openings 9 with the highly absorbent material 15 in them, and the second continuous web 104.

If the absorbent pad 4 comprises the fourth layer 16 interposed between the third layer 8 and the first bottom layer 5, the method comprises a step of positioning a fourth continuous web 121 between the third web 105, having the openings 9, and the first continuous web 101.

Advantageously, according to this invention, the third web 105 having the openings 9 in it to be interposed between the first and the second continuous web 101 and 104 can be made in-process, thus simplifying the method for making the fluffless absorbent pads 4 and considerably limiting their cost.

1. A method for making absorbent sanitary articles which comprise a permeable inner layer, an impermeable outer layer and an absorbent pad enclosed between them; the absorbent pad comprises a first bottom layer and a second top layer between which an absorbent core is enclosed; the method comprises a step of forming the absorbent pad; the step of forming the absorbent pad comprises a step of feeding a first continuous web, which will define the first bottom layer and a second continuous web, which will define the second top layer and a step of interposing between the first web and the second web at least a third web which has a plurality of openings and which will define the absorbent core; the method wherein it comprises a step of feeding a third continuous web along a feed direction and a step of cutting the third web to obtain the openings.

2. The method according to claim 1, comprising cutting the third continuous web at least along a direction parallel to the feed direction of the third continuous web to make a third cut web.

3. The method according to claim 1, comprising cutting the third continuous web at least along a direction transversal to the feed direction of the third continuous web to make a third cut web.

4. The method according to claim 1, comprising cutting defined portions of web from the third continuous web and removing these portions to make the openings of the third web.

5. The method according to claim 1, comprising a step of stretching the third continuous cut web while making the openings of the third web.

6. The method according to claim 5, wherein the step of stretching the third continuous cut web is performed at least along a direction parallel to the feed direction of the third continuous web.

7. The method according to claim 5, wherein the step of stretching the third continuous cut web is performed at least along a direction transversal to the feed direction of the third continuous web.

8. The method according to claim 5, wherein the step of stretching the third continuous cut web is performed at least along a direction parallel and transversal to the feed direction of the third continuous web.

9. The method according claim 1, comprising a step of applying a layer of adhesive substance at least on the first continuous web and a step of positioning the third web having the plurality of openings on the layer of adhesive substance applied to the first continuous web.

10. The method according to claim 1, comprising a step of applying a layer of adhesive substance at least on the second continuous web and a step of positioning the third web having the plurality of openings on the layer of adhesive substance applied to the second continuous web.

11. The method according to claim 1, comprising a step of depositing at least one highly absorbent material in the openings of the third web and a step of positioning the second continuous web on the third web where the openings comprise the highly absorbent material.

12. The method according to claim 1, comprising a step of joining at least the first and the second continuous web to the third web having the openings interposed between the first and the second continuous web.

13. The method according to claim 1, comprising a step of positioning a fourth continuous web between the third web, with the highly absorbent material in its openings, and the second continuous web.

14. The method according to claim 1, comprising a step of positioning a fourth continuous web between the third web, having the openings, and the first continuous web.
